(19) United States
(12) Patent Application Publication
(10) Pub. No.: US 2025/0312572 A1
(43) Pub. Date: Oct. 9, 2025

(54) Externally Actuatable In-Graft Catheter Fixation

(71) Applicant: Abiomed, Inc., Danvers, MA (US)

(72) Inventors: Christopher Sheils, Danvers, MA (US); Vincent Roy, Danvers, MA (US)

(73) Assignee: Abiomed, Inc., Danvers, MA (US)

(21) Appl. No.: 19/087,712

(22) Filed: Mar. 24, 2025

Related U.S. Application Data
(60) Provisional application No. 63/575,120, filed on Apr. 5, 2024.

(51) Int. Cl. A61M 25/06; A61F 2/958; A61M 39/06

(52) U.S. Cl. CPC A61M 25/0662; A61F 2/958; A61M 39/06; A61M 2039/062

(57) ABSTRACT

An assembly includes a catheter surrounded by sheath for delivering a medical device into a cardiovascular system of a patient and a catheter fixation device at least partially deployable from the sheath and transitionable between a radially collapsed state and a radially expanded state. When the catheter fixation device is in the radially collapsed state, the catheter is movable through a conduit of a vessel, and when the catheter fixation device is deployed from the sheath and expanded to the radially expanded to be in contact with an inner wall of the vessel, the catheter is fixed relative to the conduit and relative to the cardiovascular system of the patient. A method of delivering and stabilizing a medical device within a cardiovascular system of a patient is also provided.

### CROSS-REFERENCE TO RELATED APPLICATIONS

This application claims the benefit of the filing date of U.S. Provisional Patent Application No. 63/575,120 filed Apr. 5, 2024, the disclosure of which is hereby incorporated herein by reference.

### BACKGROUND OF THE DISCLOSURE

The present disclosure relates to catheter fixation devices, and more particularly, to fixation devices that allow a physician to efficiently reposition and stabilize a catheter within a vessel and relative to the cardiovascular system of a patient.

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the IMPELLA® family of devices designed by Abiomed, Inc. of Danvers, MA. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY OF THE DISCLOSURE

In accordance with a first aspect of the present disclosure, externally actuatable catheter fixation devices are provided. Among other advantages, the catheter fixation devices are designed to be delivered into a vascular graft, artery, or vein and then secured therein to subcutaneously secure a catheter carrying an intracardiac pump. Securing the catheter subcutaneously reduces the distance between the securement device and the target site (e.g., across a native heart valve) compared to securement devices which are externally secured to skin of the patient. As a result, the catheter fixation devices disclosed herein, reduce the length between the fixation point and the distal end of the catheter, which in turn reduces the likelihood of migration of the catheter compared to external securement devices. Furthermore, the externally actuatable nature of the catheter fixation devices improves useability, allowing the devices to be efficiently transitioned between secured and unsecured conditions when repositioning is desired compared to other subcutaneous catheter fixation devices.

In one embodiment, an assembly may include a catheter having a sheath for delivering a medical device into a cardiovascular system of a patient, and a catheter fixation device at least partially deployable from the sheath. The catheter fixation device may be transitionable between a radially collapsed state and a radially expanded state such that when the catheter fixation device is in the radially collapsed state, the catheter is movable through a conduit of a vessel, and when the catheter fixation device is deployed from the sheath and expanded to the radially expanded state to be in contact with an inner wall of the vessel, the catheter is fixed within the conduit and relative to the cardiovascular system of the patient.

The catheter fixation device may be a stent having a proximal end and distal end. The stent may be attached to the catheter at a location spaced from the distal end. In one example, the stent may be a self-expanding stent arranged to transition from the radially collapsed state to the radially expanded state when deployed from the sheath. Alternatively, the stent may be expandable by an inflatable balloon, or one or more springs. In some embodiments, the assembly may also include a protective ring configured to surround at least a portion of an external surface of the vessel to protect the vessel and/or surrounding anatomy when the fixation device is expanded to contact the vessel.

In another embodiment, an assembly may include a vascular graft having a proximal end, a distal end, and a sidewall defining a conduit extending between the proximal end and the distal end, and a catheter fixation ring integrated into a graft. The catheter fixation ring may be transitionable between a natural state, in which a catheter delivering a medical device is movable through the conduit, and a collapsed state, in which the catheter fixation ring clamps the catheter to fix the catheter relative to the vascular graft. In some examples, the catheter fixation ring may be formed of a resilient material and/or a magnetic material.

A method of stabilizing a medical device within a cardiovascular system of a patient is also provided herein, the method may include: tracking a catheter, carrying the medical device, through a conduit of a vessel to a target site within the cardiovascular system of the patient; and actuating an actuator, provided externally of the patient, to transition a catheter fixation device from an unsecured state, in which the catheter is movable through the conduit of the vessel, to a secured state, in which the catheter is subcutaneously fixed within the vessel and relative to the cardiovascular system of the patient.

The method may further include placing a protective ring about an external surface of the vessel at a location corresponding to the catheter fixation device. In one example, the placing step may be performed prior to the actuating step to protect the vessel and surrounding tissue when the catheter fixation device is expanded into contact with the wall of the vessel.

In some aspects, the catheter fixation device may include a self-expanding stent, and the actuating step may include deploying the self-expanding stent from a sheath surrounding the catheter and allowing at least a portion of the self-expanding stent to engage an internal surface of the vessel.

In some embodiments, the catheter may include a plurality of springs, and the actuating step may include deploying the plurality of springs and forcing the catheter fixation device to engage an internal surface of the vessel. In other embodiments, the catheter fixation device may include a balloon expandable stent, and the actuating step may include inflating a balloon and expanding the balloon expandable stent into a secure engagement with an internal surface of the vessel.

In other embodiments, the catheter fixation device may include an inflatable balloon, and the actuating step may include inflating the inflatable balloon into a secure engagement with an internal surface of the vessel.

In yet another embodiment, the catheter fixation device may include a catheter fixation ring integrated into a vascular graft. In one example, the catheter fixation ring may be formed of a resilient material, and the actuating step may include collapsing the catheter fixation ring from an unsecured state to a secured state, in which the catheter fixation ring clamps a portion of the catheter. In some embodiments, the catheter fixation ring may be formed of, or coated with, a magnetic material, and the actuating step may include delivering an electromagnet through the conduit of the vessel to an axial location corresponding to and disposed within the catheter fixation ring. In one example, the medical device may include an intracardiac pump and the target site may be across a native heart valve.

Still yet, in another embodiment, an assembly may include a catheter for delivering an intracardiac pump through a conduit of a vessel and into a heart of a patient. The catheter may include a balloon having a radially collapsed state and a radially expanded state, whereby when the balloon is in the radially collapsed state, the catheter is movable through the conduit of the vessel, and when the balloon is in the radially expanded state and engaged with an inner wall of the vessel, the catheter is stabilized within the vessel and relative to a cardiovascular system of the patient.

### BRIEF DESCRIPTION OF THE DRAWINGS

Various embodiments of the present disclosure are described herein with reference to the drawings, wherein:

FIG. 1 is a highly schematic cutaway view of the human heart, showing a percutaneous axillary artery puncture approach for delivering an intracardiac heart pump across the aortic valve;

FIG. 2 is perspective view of an intracardiac heart pump and sheath assembly according to an embodiment of the present disclosure;

FIG. 3 is a highly schematic cutaway view of the human showing the intracardiac heart pump of FIG. 2 properly positioned across the native aortic valve;

FIG. 4 is a perspective view of a catheter fixation device including a self-expanding stent according to an embodiment of the present disclosure;

FIG. 5 is a perspective view of a catheter fixation device including an expandable stent actuated by springs according to another embodiment of the present disclosure;

FIG. 6 is a perspective view of a catheter fixation device including an inflatable balloon according to yet another embodiment of the present disclosure; and

FIG. 7 is a perspective view of a catheter fixation device including a compressible catheter fixation ring integrated into a vascular graft according to a further embodiment of the present disclosure.

### DETAILED DESCRIPTION

Intracardiac pump assemblies can be introduced into the heart either surgically or percutaneously and used to pump blood from one location in the heart or circulatory system to another location in the heart or circulatory system. When deployed in the heart, for example, an intracardiac pump can transfer blood from the left ventricle to the aorta, or from the inferior vena cava to the pulmonary artery. Traditionally, intracardiac pumps operate in parallel with the native heart to supplement cardiac output and partially or fully unload components of the heart. For this reason, intracardiac pumps are often utilized in instances of cariogenic shock, during high-risk percutaneous coronary intervention (PCI), right heart failure, congestive heart failure, or severe lung failure, to relieve stress on the heart during recovery or while the patient awaits a heart transplant. Examples of such systems include the IMPELLA® family of devices designed by Abiomed, Inc., Danvers Mass.

Several IMPELLA® devices are of relatively small circumferential size and can be percutaneously delivered into a patient less invasively than intracardiac pumps that are implanted during traditional, full open-chest surgery. When delivered percutaneously, these intracardiac pumps may be inserted into a patient and delivered via a tube-like delivery device such as a catheter.

During delivery, the catheter is tracked through the vasculature of a patient to advance the intracardiac pump to a target site (e.g., across the aortic valve in the case of “left-side” intracardiac devices, or across the pulmonary valve in the case of “right-side” intracardiac devices). Echocardiography, fluoroscopy, and/or other imaging techniques may be utilized during delivery to properly position the intracardiac pump. Once in position, the intracardiac pump may be turned on to suction blood through an inflow portion (e.g., a blood inflow cage) of the intracardiac device, and to expel the pumped blood from an outflow portion (e.g., a blood outflow cage) of the intracardiac device. In the case of left-side intracardiac pumps, the blood inflow cage of the intracardiac device may be positioned within the left ventricle of the heart, and the blood outflow cage of the intracardiac pump may be positioned within the aorta. On the other hand, in the case of right-side intracardiac pumps, the blood inflow cage of the intracardiac device may be positioned within the inferior vena cava, and the blood outflow cage of the intracardiac device may be positioned within the pulmonary artery.

The clinical success of intracardiac pumps is dependent, in part, on proper positioning of the pump. For example, in the case of left-side intracardiac pumps, if the blood inflow cage is not properly positioned within the left ventricle, and without obstruction from anatomical structures such as the ventricular wall or the mitral valve, the intracardiac pump will inefficiently suction blood from the left ventricle. Additionally, if the blood outflow cage of the intracardiac pump is not sufficiently positioned within the aorta, the pumped blood may return to the left ventricle, causing further stress on the heart. Thus, it is important for intracardiac pumps to be stabilized in the proper position throughout the entire treatment. For this reason, delivery devices used in percutaneous intracardiac pump procedures, often include a butterfly structure that can be secured to the skin of a patient and a securement device, such as a conventional Tuohy-Borst type device, to prevent the catheter from moving after the intracardiac pump has been properly positioned.

Despite the improvements that have been made to intracardiac pumps and associated delivery assemblies, shortcomings remain. For example, the inventors have recognized that even when the assembly is sutured or otherwise secured to the skin of the patient, ambulation or other movement of the patient, can alter the position of the catheter relative to the anatomy of the patient and/or the access site, and cause the intracardiac pump to migrate from the desired position (i.e., become mispositioned). In some instances, such movement may even damage or completely break the sutures, resulting in further movement of the unsecured catheter. Accordingly, the inventors have recognized and appreciated the numerous benefits associated with subcutaneously securing a catheter within a graft (or the native vasculature) to reduce migration of the catheter relative to cardiovascular system of the patient and/or the access site, while retaining the user-friendly nature of traditional externally actuatable catheter fixation devices.

When deployed in the heart, an intracardiac pump collects blood from one area of the heart and pumps the blood to another area of the heart, to assist the heart in performing its normal function. As used herein in connection with an intracardiac pump, the term “inflow” refers to the portion of the intracardiac pump through which blood enters the pump, and the term “outflow” refers to the portion of the intracardiac pump through which blood is expelled. When used in connection with devices for delivering the intracardiac pump into a patient, the terms “proximal” and “distal” are to be taken as relative to the user of the delivery devices. For example, “proximal” or “proximal end” is to be understood as relatively close to the operator, and “distal” or “distal end” is to be understood as relatively farther away from the operator. Also as used herein, the terms “substantially,” “generally,” “approximately,” and “about” are intended to mean that slight deviations from absolute are included within the scope of the term so modified.

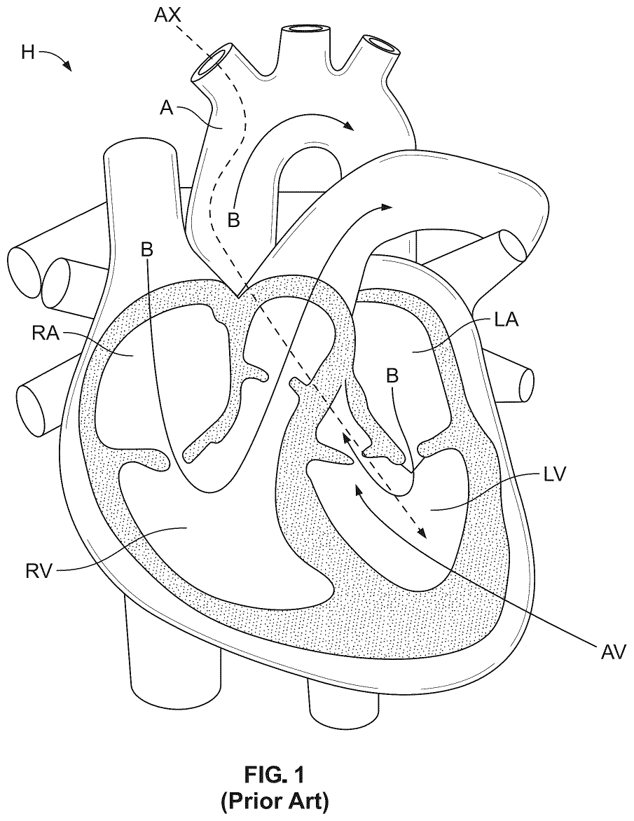

FIG. 1 is a schematic cutaway representation of a human heart H. The human heart includes two atria and two ventricles: right atrium RA and left atrium LA, and right ventricle RV and left ventricle LV. Heart H further includes aorta A. Disposed between left ventricle LV and aorta A is aortic valve AV. The aortic valve, also known as the left semilunar valve or the left arterial valve, generally includes three leaflets that coapt to regulate blood flow between left ventricle LV and aorta A. When left ventricle LV contracts during systole, aortic valve AV opens, and blood is pushed from the left ventricle through aorta A to major arteries of the vasculature system. Blood flows through heart H in the direction shown by arrows “B”.

A dashed arrow, labeled “AX”, indicates an approach of delivering an intracardiac pump to a target site via the axillary artery, in this case to a location across aortic valve AV. In such cases, an incision is made on the axillary artery and a graft is attached. The intracardiac blood pump is inserted into the graft, advanced through the axillary artery, and to the target site through the aorta A. In some embodiments, an introducer sheath may be inserted into the graft. In such embodiments, the intracardiac blood pump may pass through the introducer sheath, through the axillary artery, and to the target site through the aorta A. Echocardiography, fluoroscopy, and/or other imaging techniques may be used to help guide a delivery device, such as a catheter, to the target site. In some embodiments, the intracardiac pump may be delivered via a more proximate vessel, such as the subclavian, aortic, or innominate. Other approaches are possible for delivering the intracardiac pump across aortic valve AV, such as a direct aortic approach, or to other target sites within heart H.

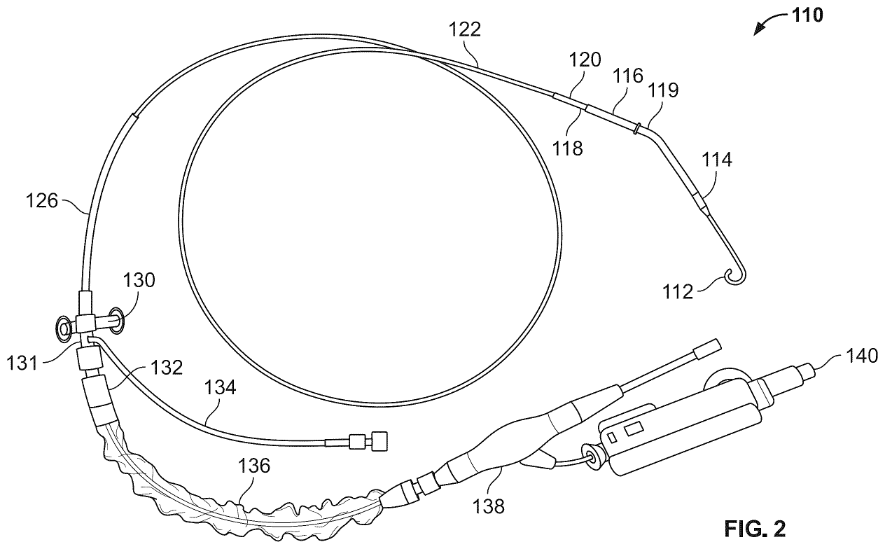

FIG. 2 illustrates an intracardiac pump and sheath assembly 110. A handle 138 may be provided at the proximal end of intracardiac pump and sheath assembly 110. Handle 138 may be operably coupled to the proximal end of a catheter 122 and, therefore, arranged to advance the catheter within the cardiovascular system of a patient and to retract the catheter from the cardiovascular system of the patient, unless movement of the catheter is prevented by a securement device 132.

Catheter 122 may be enclosed by a protective sleeve 136 extending between handle 138 and securement device 132. Put differently, the proximal end of protective sleeve 136 may be attached to handle 138, and the distal end of the protective sleeve may be attached to securement device 132, to enclose and protect a proximal portion of catheter 122. Protective sleeve 136 may be formed of any material, such as a medical grade plastic, suitable for preventing the proximal end of catheter 122 from being contaminated as the catheter is advanced into the vasculature of a patient.

A hemostasis valve 131 may be provided between a distal end of securement device 132, and a proximal end of butterfly 130. Hemostasis valve 131, securement device 132, and butterfly 130 may be removably coupled from one another or manufactured as a single unitary component. A proximal end of a sheath 126 may be coupled to butterfly 130. Sheath 126 defines a lumen extending from the proximal end of the sheath to a distal end of the sheath that is sized to slidably receive catheter 122 therethrough.

Intracardiac pump and sheath assembly 110 may also include a purge fluid port 140. The purge fluid port 140 may be in communication with handle 138 and may be arranged to provide purge fluid (e.g., a solution of dextrose or glucose with heparin) to a purge lumen (not shown) within catheter 122. The purge lumen of catheter 122 may be in fluid communication with, and configured to deliver the purge fluid to, the motor housing 120 of the intracardiac pump. Motor housing 120 may include a motor and an impeller. In some embodiments, the motor may be external to the patient, in which case catheter 122 may enclose a flexible drive, such as a shaft or cable, and motor housing 120 may enclose an impeller connected to that drive shaft or cable.

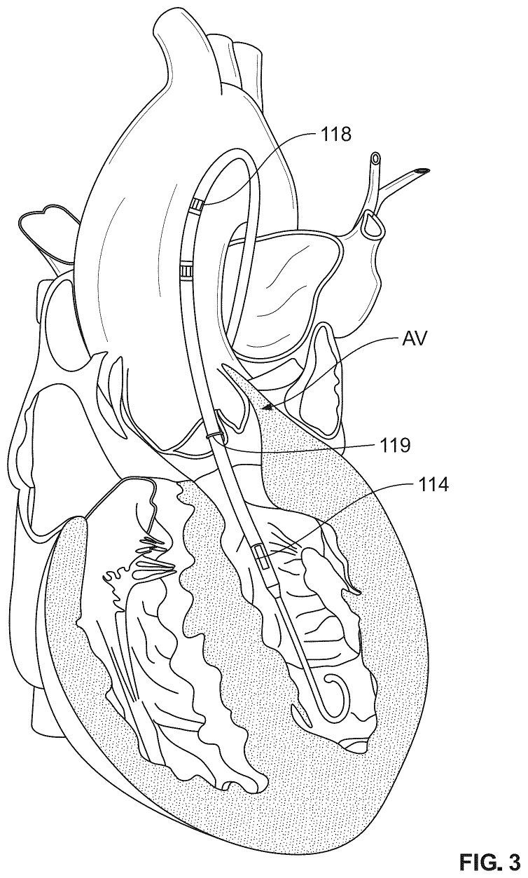

With continued reference to FIG. 2, the distal end of motor housing 120 may be coupled to a blood outflow cage 118. The distal end of blood outflow cage 118 may be coupled to a cannula 116, which in turn, may be coupled a proximal end of blood inflow cage 114. Cannula 116 may include a marking 119, such as a radiopaque marker that is visible under fluoroscopy, to assist a clinician in properly positioning the cannula within the native heart valve as shown in FIG. 3. In some embodiments, the cannula 116 may be expandable. A pigtail extension 112 may extend from the distal end of blood inflow cage 114. In this regard, when left ventricle LV contracts during systole, the left ventricular wall may contact pigtail extension 112, instead of blood inflow cage 114, thereby preventing intracardiac pump from damaging the ventricular wall. In other embodiments, the intracardiac pump may not include a pigtail extension 112.

When the pump is operated, blood will be pumped in the proximal direction from blood inflow cage 114, through cannula 116, to blood outflow cage 118. In this respect, the intracardiac pump illustrated in FIG. 2 is designed for left heart support. The intracardiac pump, however, may alternatively be configured to pump blood in the distal direction (e.g., for applications where the pump is used for right heart support), in which case cage 118 would operate as the blood inflow cage, and cage 114 would operate as the blood outflow cage.

Intracardiac pump and sheath assembly 110 may be introduced into the vasculature of a patient via an introducer sheath assembly (not shown). In some embodiments, the intracardiac pump may be inserted into vasculature of the patient through a graft sutured to the cardiovascular system of a patient, in a chimney fashion. For example, when delivering an intracardiac pump using a percutaneous axillary artery approach, intracardiac pump and sheath assembly 110 may be inserted through a graft secured to the axillary artery.

Once sheath 126 has been fully inserted into the patient, the clinician may secure the sheath to the patient at or near the incision, for example, adjacent the clavicle using butterfly 130. That is, butterfly 130 may be affixed to the patient, via adhesives or sutures, to secure the sheath 126 and securement device 132 relative to the patient. With sheath 126 secured to the patient, the clinician may then adjust the intracardiac pump relative to the target site and actuate securement device 132 to restrict further movement of the intracardiac pump after it has been properly positioned within heart H.

Securement device 132 may be a conventional Tuohy-Borst type device, although any other securement device known in the art may be used. Conventional Tuohy-Borst type devices include a barrel that may be rotated in a first direction (e.g., a clockwise direction) to clamp securement device 132 about catheter 122, which in turn restricts movement of the catheter. As mentioned herein, however, movement of the patient can adjust the positioning of catheter 122, which in turn, can cause migration of the intracardiac pump. More particularly, movement of the patient may: (1) adjust the position of catheter 122 located distal of the securement device 132 relative to the cardiovascular system of the patient; and/or (2) displace butterfly 130, or other device securing the proximal end of sheath 126 to the patient (e.g., rupturing of the sutures or peeling of the adhesive), which may cause the entire assembly to shift. When catheter 122 is mispositioned, it may be desirable to reposition the catheter to return the intracardiac pump to a desired position.

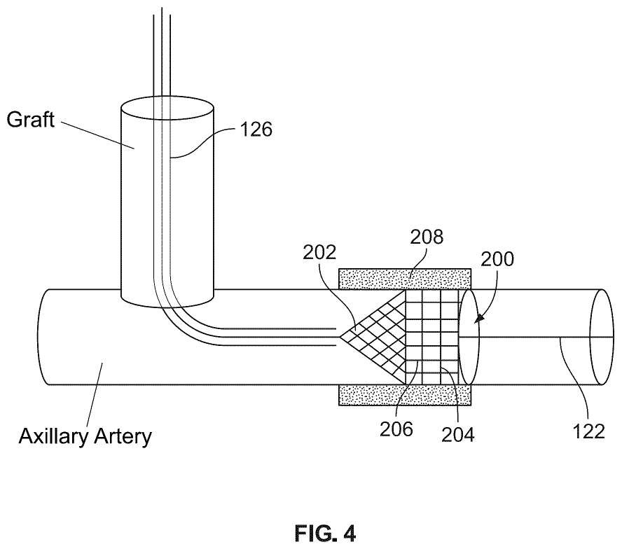

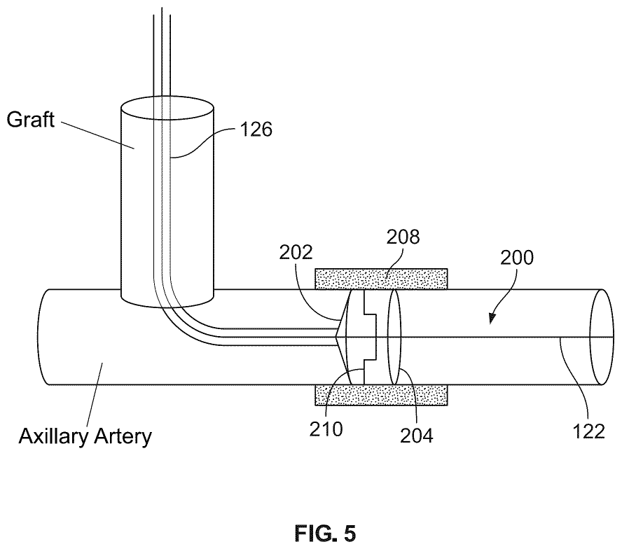

FIGS. 4 and 5 illustrate a catheter fixation device 200 according to an embodiment of the present disclosure. Catheter fixation device 200 may be transitionable between a radially collapsed state (e.g., collapsed state) and a radially expanded state (e.g., expanded state) to secure catheter 122 within a vessel conduit (i.e., within a vascular graft, a native artery, or a native vein). Catheter fixation device 200 may be actuatable by an external mechanism provided, for example, on the handle 138 of intracardiac pump and sheath assembly 110. It will be appreciated that the intracardiac pump will be more stably secured within heart H as the distance between catheter fixation device 200 and heart H decreases. That is, reducing the distance between the intracardiac pump and the location at which the catheter is fixed, will reduce the ability of the intracardiac pump to migrate. For this reason, securing catheter 122 within a vessel conduit, and proximate heart H, will improve the stability of the intracardiac pump compared to external securement devices.

Catheter fixation device 200 is primarily described herein in conjunction with intracardiac pump and sheath assembly 110. However, catheter fixation device 200 may be used with any intracardiac blood pump, or other medical device, delivered into a patient by a delivery system, such as a catheter, when a clinician wishes to selectively restrict movement of that device. Examples of such systems include angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, or any other venous or arterial based introduced catheters and devices.

It will be appreciated that catheter fixation device 200 is designed to perform a similar function to that of butterfly 130 and securement device 132 (e.g., stabilize catheter 122 relative to the cardiovascular system of the patient). Consequently, in some embodiments, catheter fixation device 200 may be used with intracardiac pump and sheath assembly 110 as depicted in FIG. 2 (i.e., along with butterfly 130 and securement device 132), while in other embodiments, catheter fixation device 200 may render the use of butterfly 130 and securement device 132 unnecessary.

With reference to FIG. 4, catheter fixation device 200 has a proximal portion and a distal portion 204. The proximal portion 202 of catheter fixation device 200 may be coupled to catheter 122 and the distal portion 204 of catheter fixation device 200 may extend distally towards motor housing 120. In some embodiments, the proximal portion 202 of catheter fixation device may be permanently affixed to catheter 122. In other implementations, the proximal portion 202 of catheter fixation device may include a retaining element (now shown) corresponding in shape and configured to be held by a retainer (not shown), for example, a recess provided on a section of catheter 122 located proximal to motor housing 120.

Catheter fixation device 200 may be formed from a network of braided wires 206 or mesh forming a stent-like device or other structure having cells. Preferably, the braided wires or mesh comprise a biocompatible material that is capable of self-expansion, for example, a shape memory alloy such as nitinol. The nitinol mesh or braid may be configured such that the proximal portion 202 of catheter fixation device 200 is substantially funnel shaped in the expanded state, and the distal portion 204 of catheter fixation device 200 is generally cylindrical in shape when expanded. Catheter fixation device 200 may be radially crimped or collapsed (e.g., toward a longitudinal axis of the catheter) to a diameter that allows it to be inserted into an annular space between catheter 122 and sheath 126 for delivery target site, and then deployed from the lumen of the sheath and radially expanded against an interior surface of a vessel. Alternatively, the braided wires or mesh may be formed of a resilient material that is not capable of self-expansion. In such instances, catheter 122 may include a mechanism such as springs 210 (FIG. 5) or an inflatable balloon provided on catheter 122 at a location corresponding to the catheter fixation device 200 to force the braided wires into the expanded state. In some embodiments, a radiopaque marker may be provided on catheter fixation device 200, for example on distal portion 204, to allow the catheter fixation device 200 to be seen under fluoroscopy and/or echocardiography.

In use, catheter fixation device 200 may be used in conjunction with an intracardiac pump and sheath assembly 110 to efficiently stabilize the intracardiac pump within heart H. Although use of catheter fixation device 200 is described hereinafter in connection with a percutaneous intracardiac pump that is delivered to the left-side of the heart using an axillary approach, it will be appreciated that the catheter fixation device 200 may be used in conjunction with percutaneous intracardiac devices providing right heart support, as well as other medical devices secured to other delivery systems for which a clinician wishes to selectively restrict movement.

First, a physician may make an infraclavicular incision to provide an access point to the axillary artery of the patient. In some aspects, a vascular graft may then be sutured to the axillary artery in a chimney fashion. In other aspects, a physician may make a supraclavicular incision to provide an access point to the subclavian and/or innominate arteries of the patient. In such aspects, a vascular graft may then be sutured to the subclavian or innominate artery. With the graft secured to the vasculature, catheter fixation device 200 may be coupled to catheter 122 (if not permanently affixed) and intracardiac pump may then be secured to the distal end of intracardiac pump and sheath assembly 110. Sheath 126 may then be slide distally over catheter fixation device 200, collapsing catheter fixation device 200 within the lumen of the sheath, and then distally over the intracardiac pump for delivery into the patient. Intracardiac pump and sheath assembly 100 may then be inserted percutaneously into the patient, through the graft.

Under echocardiography and/or other traditional imaging techniques, the clinician may advance catheter 122 toward the target site by tracking the intracardiac pump into heart H via aorta A. As shown in FIG. 3, while using fluoroscopy, the physician may align the marking 119 on cannula 116 within the aortic valve AV for deployment in the left ventricle of a patient. Once deployed, blood inflow cage 114 may be centrally positioned within left ventricle LV and blood outflow cage 118 may be sufficiently positioned within aorta A.

After the clinician has confirmed that the intracardiac pump is properly positioned within the heart H of the patient, the clinician may then deploy catheter fixation device 200 by retracting sheath 126 in a proximal direction, exposing the catheter fixation device, and enabling the braided wires 206 to radially expand such that distal portion 204 engages the surrounding vessel (e.g., the graft and/or native vasculature of the patient such as the axillary artery or aorta A) to securely stabilize catheter 122 within the vessel and relative to the cardiovascular system of a patient. In aspects in which the braided wire 206 or mesh is formed of a self-expanding material, catheter fixation device 200 will automatically expand when unsheathed. In aspects in which the braided wire 206 or mesh is not formed of a self-expanding material, the clinician may actuate an actuator in handle 138 to expand springs 210, an inflatable balloon, or another mechanism to transition catheter fixation device 200 to the expanded state.

In some embodiments, a surgeon may place a protective ring 208 around the vessel at a location corresponding to catheter fixation device 200 prior to expanding the catheter fixation device. In this regard, protective ring 208 may prevent the vessel from overly expanding due to the expansion of catheter fixation device 200, thereby more stably securing the catheter fixation device within the vessel while simultaneously protecting the vessel and surrounding anatomy from damage.

If the intracardiac pump becomes mispositioned relative to the native valve annulus during use, for example, because of movement of the patient, the physician may advance the distal end of sheath 126 over at least a portion of catheter fixation device 200, which in turn will at least partially collapse the distal portion 204 of the catheter fixation device from the vessel. The physician may then advance or retract catheter 122 to properly reposition the intracardiac pump. After the physician has determined that the intracardiac pump is positioned at correct depth across the native valve annulus, the physician may again deploy catheter fixation device, enabling the distal portion 204 of catheter fixation device to engage the vessel and stabilize catheter 122 within the vessel.

With the intracardiac pump stabilized across the aortic valve AV, the intracardiac pump may be turned on to suction blood from the left ventricle LV into blood inflow cage 114, through connecting cannula 116, and out from blood outflow cage 118 into aorta A. Thereafter, the incision may then be closed. While FIGS. 4 and 5 depict catheter fixation device 200 secured within the axillary artery, it will be appreciated that the catheter fixation device may be used to secure catheter 200 within the graft (if used), aorta A, or any other vessel within the cardiovascular system of the patient.

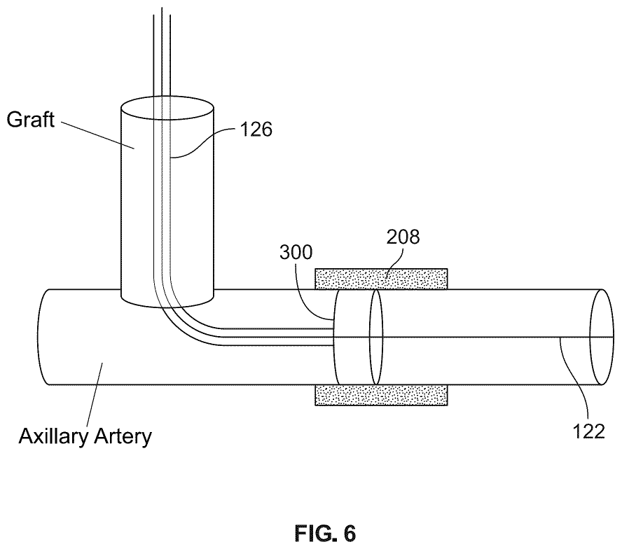

Turning now to FIG. 6, a catheter fixation device 300 according to another embodiment of the present disclosure is provided. As depicted in FIG. 6, catheter fixation device 300 may be a balloon capable of being inflated by an actuator button located, for example, in handle 138. The balloon of catheter fixation device 300 may be generally cylindrical in shape although the disclosure is not limited thereto. In some embodiments, the actuator may be a hydraulically actuator, or a fluid pump, in communication with the balloon via an inflation channel (not shown) extending through catheter 122. In this regard, the balloon of catheter fixation device 300 is transitionable between a collapsed state in which catheter 122 is movable within the vessel, and an expanded state in which the balloon expands into an engagement with an interior surface of the vessel to secure catheter 122 therein.

In use, a surgeon may operate catheter fixation device 300 to securely stabilize intracardiac pump and sheath assembly 110 within heart H in the same manner as previously described with respect to the embodiments referenced in FIGS. 4 and 5 except for the manner in which the catheter fixation device is inflated. For this reason, only this aspect of the procedure will be described hereinafter.

After the clinician has confirmed that the intracardiac pump is properly positioned within the heart H of the patient, the physician may press or otherwise actuate an actuator to inflate the balloon and stabilize catheter 122 within the vessel and relative to the cardiovascular system and/or the access of the patient. It will be appreciated that the actuator button is merely exemplary and that any externally actuated actuator may be used to inflate the balloon. In the event that the surgeon later desires to reposition catheter fixation device 300, the balloon may be deflated before the intracardiac pump is repositioned relative to the vessel. After catheter fixation device 300 has been repositioned, the physician may then again inflate the balloon to secure the catheter relative to the cardiovascular system of the patient.

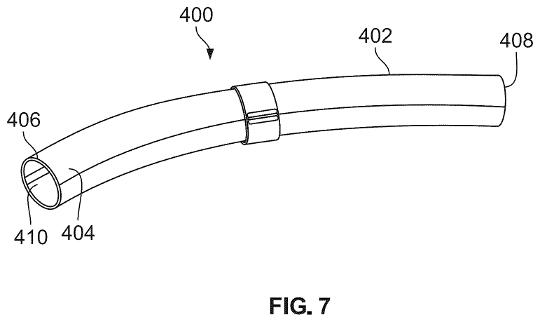

With reference to FIG. 7, one or more catheter fixation devices 400 may be integrated into a graft 402 formed of a biocompatible material, such as a woven fabric, for example, Dacron or polytetrafluoroethylene. However, it will be appreciated that any other materials known in the vascular grafting art may be utilized. Graft 402 may be generally tubular in shape and include a sidewall 404 extending from a proximal end 406 to a distal end 408 that defines a conduit 410 sized and configured to receive intracardiac pump and sheath assembly 110. The distal end 408 of graft 404 may be designed to be sutured to the axillary artery in a chimney fashion.

Catheter fixation devices 400 may be integrated into graft 402 and configured to transition from a natural state, in which catheter 122 is movable along the length of the conduit, to collapsed state to clamp the catheter and secure the catheter within the conduit 410 of graft 402 and relative to the cardiovascular system of the patient. As used herein, “integrated” or “integrally formed” means the one or more catheter fixation devices 400 are pre-operatively coupled to graft 402, for example, during manufacturing of the graft, or by a user, after the graft is manufactured and prior to the graft being sutured to the cardiovascular system of the patient.

As depicted in FIG. 7, catheter fixation device 400 may be formed as a catheter fixation ring that extend continuously about a circumference of conduit 410. For example, the catheter fixation ring may be coupled about an external surface of the sidewall 404, an internal surface of the sidewall, or form a portion of the sidewall of graft 402. In other embodiments, the catheter fixation ring may discontinuously extend about a circumference of conduit 410 such that the combination of the catheter fixation ring and sidewall 404 form a portion of the conduit. In some embodiments, the thickness of the catheter fixation ring may be equal to the thickness of the sidewall 404. In other embodiments, the thickness of the catheter fixation ring may be different than the thickness of the sidewall 404 graft 402. For example, the thickness of the catheter fixation device 400 may be greater than the thickness of the sidewall 404 of graft 402.

Catheter fixation device 400 may, for example, be formed of a substantially resistant material, such as a polymer, and more particularly a rubber or a silicone. In this regard, catheter fixation device 400 may be designed to compress catheter 122 while distributing the compressible load to prevent damage. However, it will be appreciated that catheter fixation ring, may be formed of any suitable material. In some examples, an innermost surface of catheter fixation device 400 may include, or be coated with, a magnetic material. In this regard, the catheter fixation ring may be configured to transition to the collapsed state when an electromagnet is disposed and activated within the catheter fixation ring. The electromagnet may be provided on catheter 122 itself, or alternatively, on different delivery device such as another catheter or guidewire. In other embodiments, the catheter fixation ring may be transitioned between its natural state and its collapsed state using another mechanical, magnetic, electromagnetic, or wireless mechanism.

In use, a surgeon may operate catheter fixation device 400 to securely stabilize intracardiac pump and sheath assembly 110 within heart H in a similar manner to catheter fixation devices 200, 300 except for the manner in which the catheter fixation device is collapsed about catheter 122. Thus, for brevity, only this aspect of the procedure will be described hereinafter.

Once the surgeon has confirmed that the intracardiac pump is properly positioned within the heart H of the patient, the physician may position an electromagnet within the ring of catheter fixation device 400. In some embodiments, the surgeon may perform this step by aligning an electromagnet provided on catheter 122 within the ring, while in other embodiments the surgeon may delivery another device carrying the electromagnet through graft 402 and into position within the catheter fixation ring. Thereafter, the surgeon may activate the electromagnet, for example, by activating a button provided on the handle 138 of intracardiac pump and sheath assembly 110 to produce a magnetic field which will attract catheter fixation device 400 and cause it to collapse about catheter 122, thereby securing the catheter within the graft and relative to the cardiovascular system of the patient. Of course, the electromagnetic actuator is merely exemplary and one of ordinary skill in the art may easily derive other means for mechanically, magnetically, or wirelessly collapsing catheter fixation device 400 about catheter 122 without departing from the scope of the disclosure.

If the event that the surgeon later desires to reposition catheter 122, the surgeon may deactivate the electromagnet, which in turn which break the magnetic connection between the electromagnet and the catheter fixation ring and cause the catheter fixation ring to expand in size as it returns to its normal state. After the catheter fixation ring has expanded, the surgeon may freely adjust the position of catheter 122 to a desired location, before again actuating the electromagnet, which will cause the catheter fixation ring to collapse about catheter 122 as previously described.

To summarize the foregoing, catheter fixation devices 200, 300, 400 offer several advantageous over traditional external securement devices. For example, when catheter 122 is externally secured, movement of the patient may result in movement of the catheter, which in turn, may result in migration of the intracardiac pump. Catheter fixation devices 200, 300, 400 permit catheter 122 to be stabilized subcutaneously (e.g., closer to the heart) which reduces catheter 122 motion due to patient movement. Furthermore, catheter fixation devices 200, 300, 400 are externally actuatable, thereby providing a surgeon with the ability to selectively stabilize catheter 122 in position and reposition the catheter when an adjustment in is desired.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An assembly, comprising:
a catheter for delivering a medical device into a cardiovascular system of a patient, the catheter having a sheath; and
a catheter fixation device at least partially deployable from the sheath and transitionable between a radially collapsed state and a radially expanded state,
wherein when the catheter fixation device is in the radially collapsed state, the catheter is movable through a conduit of a vessel, and when the catheter fixation device is deployed from the sheath and expanded to the radially expanded state to be in contact with an inner wall of the vessel, the catheter is fixed within the conduit and relative to the cardiovascular system of the patient.

2. The assembly of claim 1, wherein the catheter fixation device is a stent having a proximal end and distal end.

3. The assembly of claim 2, wherein the stent is attached to the catheter at a location spaced from the distal end.

4. The assembly of claim 2, wherein the stent is a self-expanding stent arranged to transition from the radially collapsed state to the radially expanded state when deployed from the sheath.

5. The assembly of claim 2, wherein the stent is expandable by an inflatable balloon.

6. The assembly of claim 2, wherein the stent is expandable by one or more springs.

7. The assembly of claim 1, further comprising a protective ring configured to surround at least a portion of an external surface of the vessel.

8. An assembly, comprising:
a vascular graft having a proximal end, a distal end, and a sidewall defining a conduit extending between the proximal end and the distal end; and
a catheter fixation ring integrated into a graft, the catheter fixation ring being transitionable between a natural state, in which a catheter delivering a medical device is movable through the conduit, and a collapsed state, in which the catheter fixation ring clamps the catheter to fix the catheter relative to the vascular graft.

9. The assembly of claim 8, wherein the catheter fixation ring comprises a resilient material.

10. The assembly of claim 8, wherein the catheter fixation ring comprises a magnet.

11. A method of stabilizing a medical device within a cardiovascular system of a patient, the method comprising:
tracking a catheter, carrying the medical device, through a conduit of a vessel to a target site within the cardiovascular system of the patient;
actuating an actuator, provided externally of the patient, to transition a catheter fixation device from an unsecured state, in which the catheter is movable through the conduit of the vessel, to a secured state, in which the catheter is subcutaneously fixed within the vessel and relative to the cardiovascular system of the patient.

12. The method of claim 11, further comprising:
placing a protective ring about an external surface of the vessel at a location corresponding to the catheter fixation device.

13. The method of claim 12, wherein the placing step is performed prior to the actuating step.

14. The method of claim 11, wherein the catheter fixation device comprises a self-expanding stent, and the actuating step comprises:
deploying the self-expanding stent from a sheath surrounding the catheter and allowing at least a portion of the self-expanding stent to engage an internal surface of the vessel.

15. The method of claim 11, wherein the catheter comprises a plurality of springs, and the actuating step comprises:
deploying the plurality of springs and forcing the catheter fixation device to engage an internal surface of the vessel.

16. The method of claim 11, wherein the catheter fixation device comprises a balloon expandable stent, and the actuating step comprises:
inflating a balloon and expanding the balloon expandable stent into a secure engagement with an internal surface of the vessel.

17. The method of claim 11, wherein the catheter fixation device comprises an inflatable balloon, and the actuating step comprises:
inflating the inflatable balloon into a secure engagement with an internal surface of the vessel.

18. The method of claim 11, wherein the catheter fixation device comprises a catheter fixation ring integrated into the vessel comprising a vascular graft, the catheter fixation ring comprising a resilient material, and the actuating step comprises:
collapsing the catheter fixation ring from the unsecured state, in which the catheter fixation ring is in a natural state, to the secured state, in which the catheter fixation ring clamps the catheter.

19. The method of claim 18, wherein the catheter fixation ring comprises a magnetic material, and the actuating step comprises:
delivering an electromagnet through the conduit of the vessel to an axial location within the catheter fixation ring.

20. The method of claim 11, wherein the medical device comprises an intracardiac pump and the target site is across a native heart valve.
